(19) United States
(12) Patent Application Publication
(10) Pub. No.: US 2009/0062614 A1
(43) Pub. Date: Mar. 5, 2009

(54) CLOSURES FOR OUTER TUBE FOR NATURAL ORIFICE SURGERY

(75) Inventors: VASO ADZICH, Santa Ana, CA (US); Wayne A. Noda, Mission Viejo, CA (US); Stephen Graham Bell, Roma (IT); Elbert Y. Tzeng, Irvine, CA (US)

Correspondence Address: ROGITZ & ASSOCIATES, 750 B STREET, SUITE 3120, SAN DIEGO, CA, 92101, US

(73) Assignee: MINOS MEDICAL

(21) Appl. No.: 12/164,557

(22) Filed: Jun. 30, 2008

Related U.S. Application Data
(63) Continuation-in-part of application No. 11/846,730, filed on Aug. 29, 2007.
(60) Provisional application No. 61/024,180, filed on Jan. 28, 2008.

(51) Int. Cl. A61B 1/31

(52) U.S. Cl. 600/129

(57) ABSTRACT

A plug is closely received in the open distal end of an outer tube for natural orifice surgery and is proximally removable from the tube. Various means are provided for deforming the plug to permit the plug to be retrieved through the outer tube.

This is a continuation-in-part of U.S. patent application Ser. No. 11/846,730, filed Aug. 29, 2007, from which priority is claimed. This application also claims priority from U.S. provisional application 61/024,180, filed Jan. 28, 2008.

### I. FIELD OF THE INVENTION

The present invention relates generally to systems and methods for transanal and transoral surgical procedures such as for the resolution of appendicitis, gall bladder maladies, and diverticula or performing polypectomy and esophagogastroduodenscopy.

### II. BACKGROUND OF THE INVENTION

In the present assignee's U.S. patent applications Ser. Nos. 11/601,199, 11/606,742, and 11/788,597, all of which are incorporated herein by reference, devices and methods are disclosed for natural orifice procedures. Specifically, devices and methods are disclosed in which a medical instrument is advanced through the mouth or anus of a patient to resolve maladies with organs such as polyps, the gall bladder and appendix, and to treat diverticulosis. As understood herein, such procedures can be facilitated by using a relatively large outer tube through which one or more instruments such as endoscopes, e.g., colonoscopes, may be advanced to facilitate the desired procedure. It is to such outer tubes that the present invention is directed.

### SUMMARY OF THE INVENTION

An apparatus for engaging an end of a tube includes a plug having a body and a spiral score on an outer surface of the body to facilitate material separation therealong. A disruption tool is configured to engage the body to cause material separation along the spiral score. In some embodiments the disruption tool can include a hook embedded in the body and graspable by a snare to impart tensile force to cause the material separation. The tube may be an outer tube configured to slidably bear instruments for natural orifice surgery and the plug may fit snugly in the end of the tube.

In another aspect, an assembly includes an outer tube configured to bear one or more instruments for natural orifice surgery. The outer tube defines an open distal end. A plug is closely received in the open distal end and is proximally removable therefrom. Means are provided for deforming the plug to permit the plug to be retrieved through the outer tube.

In some embodiments the means for deforming includes a rigid hook pullable to cause the plug to tear. In non-limiting implementations the plug includes a body having a first portion materially weaker than a second portion of the body. The first portion can include a wall defining a first thickness, and the second portion can include a wall defining a second thickness greater than the first thickness. The first portion may include at least one score that may be spiral and that may be formed on an inside surface of the body or on an outside surface of the body.

In other embodiments the plug can be formed with a flange-like retention tongue proximal to a distal tip portion of the plug, and the tongue can be received in a groove of a tip ring positioned at the distal end of the outer tube.

In other embodiments the plug can be formed with a main body portion defining a cylinder with axial cutout and a keystone portion fitting snugly in the cutout. The keystone portion can be removable from the cutout to facilitate radially collapsing the main body portion to permit removal thereof through the outer tube.

In other embodiments the plug can include plural axial segments joinable to define a cylindrical configuration. The segments can be connected together by a connector and being separable from each other to facilitate removal of the segments through the outer tube.

In other embodiments the plug is formed with plural perforations to facilitate deforming the plug.

In other embodiments the plug can be an inflatable balloon positioned on an endoscope deliverable through the outer tube.

In other embodiments the plug can be an expandable mesh positioned on an endoscope deliverable through the outer tube.

In other embodiments the plug can be an inner body closely received in the outer tube at a distal end thereof. The body may be deformable and cylindrical and/or the body may be deformable foam and/or the body may define a tapered distal segment.

Any of the plugs herein may be hollow to receive, e.g., an endoscope or solid to completely close the distal end of the outer tube.

In another aspect, an assembly for natural orifice surgery includes an elongated flexible outer tube defining a single main lumen and an open distal end. A distal flexible nose tube surrounds the outer tube. The nose tube is movable from a blocking configuration, wherein the nose tube extends beyond the open distal end of the outer tube and at least a portion of the nose tube extends radially inwardly from a wall of the outer tube, and a retracted configuration, wherein substantially the entire nose tube is positioned flush against an outer wall of the outer tube.

In another aspect, an assembly for natural orifice surgery includes an elongated flexible outer tube defining a single main lumen and an open distal end. An iris-type distal tip surrounds the outer tube. The tip is movable from a blocking configuration, wherein the tip extends beyond the open distal end of the outer tube and at least a portion of the tip extends radially inwardly from a wall of the outer tube, and a retracted configuration, wherein substantially the entire tip is positioned flush against an outer wall of the outer tube.

In another aspect, an assembly for natural orifice surgery includes an elongated flexible outer tube defining a single main lumen and an open distal end. A connector stub is removably engageable with a proximal end of the outer tube coaxially therewith. Also, an elongated extension tube is removably connected to the stub coaxially therewith to add working length to the assembly.

In another aspect, an assembly for natural orifice surgery includes an elongated flexible outer tube defining a single main lumen. An insert is slidably engageable with the main lumen and defines one or more sub-lumens. The insert substantially radially fills the main lumen. The sub-lumen configured for slidably receiving an endoscope. An axially rigid stiffener is engageable with the insert to push the insert into the main lumen.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view showing the outer tube, an insert holding medical instruments, and a distal plug, with a replacement insert shown nearby the outer tube;

FIG. 2 is a cross-section as seen along the line 2-2 in FIG. 1;

FIG. 3 is a cross-section as seen along the line 3-3 in FIG. 1,

FIGS. 4 and 5 are side views of two embodiments of the distal plug, also showing, through the transparent outer tube, a tether attached to each plug, with the plug in FIG. 4 shown pushed part way out of the tube;

FIG. 6 is a perspective view of the proximal end of an alternate overtube having a proximal plug with handle instead of an insert, with portions of the replacement insert broken away for clarity;

FIGS. 7 and 8 show an alternate balloon-implemented distal plug in the inflated and deflated configuration, respectively;

FIG. 9 is a side view of the distal end of an alternate outer tube, with a vacuum ring formed around the periphery of the open distal end;

FIG. 10 is a cut-away perspective view of the outer tube, showing an insert completely advanced into the tube through the proximal handle shown in FIG. 6;

FIG. 11 is a cut-away perspective view of the proximal portion of the outer tube, showing an insert being advanced into the tube to illustrate the longitudinal ribs and slit of the non-limiting insert;

FIG. 12 is a cut-away perspective view showing the distal end of a forward-viewing endoscope;

FIG. 13 is a cut-away perspective view showing the distal end of a side-viewing endoscope;

FIG. 14 is a perspective view of an alternate distal plug that can be retrieved by tearing the plug and retrieving it through the overtube, with the embedded hook omitted for clarity;

FIG. 15 is a side view of the plug shown in FIG. 14, showing the hook;

FIGS. 16 and 17 are partial cross-sectional views illustrating an alternate tear-away plug being retrieved through an overtube;

FIG. 18 is a cross-sectional view of an overtube with distal plug and retention ring;

FIGS. 19-21 are perspective views showing an alternate tear-away plug;

FIGS. 22 and 23 are perspective views showing another alternate tear-away plug;

FIGS. 24 and 25 are perspective views showing yet another alternate tear-away plug;

FIG. 26 is a perspective view illustrating a stiffener for installing the multi-channel insert in the overtube;

FIGS. 27-29 illustrate an introducer balloon that can be used to close the end of an overtube;

FIGS. 30 and 31 illustrate a frame that can be used in lieu of the balloon shown in FIGS. 27-29;

FIG. 32 is a perspective view showing an overtube extension assembly;

FIGS. 33 and 34 show a retractable elastic nosepiece for an overtube;

FIGS. 35-37 illustrate an iris-type foldable overtube tip;

FIGS. 38 and 39 illustrate an interior removable overtube tip; and

FIG. 40 is a cross-sectional view showing a deformable foam tip for an overtube.

### DETAILED DESCRIPTION OF THE PREFERRED EMBODIMENT

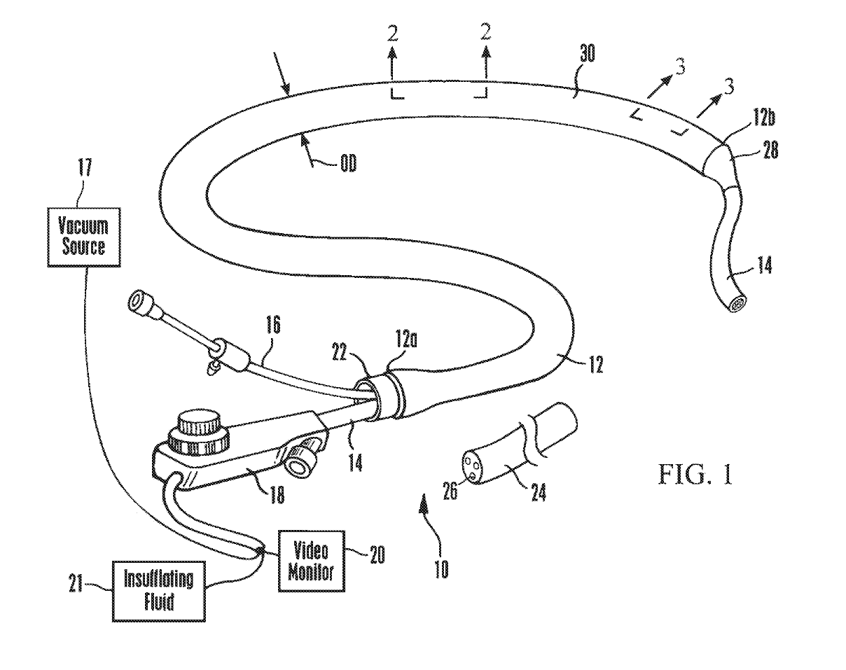

Referring initially to FIG. 1, an assembly is shown, generally designated 10, that includes a flexible hollow outer tube 12 fixedly or slidably holding one or more components such as but not limited to an endoscope 14 such as a colonoscope and an ancillary catheter 16, either one of which catheters may be connected to a source 17 of vacuum. The endoscope 14 may extend from the open distal end 12b of the outer tube 12 as shown to an endoscope control hub 18 that is external to the patient. In this way, for example, images of the colon from the endoscope 14 can be presented on a monitor 20 to a surgeon. Additional components, e.g., a source 21 of insufflating fluid, may extend through or be engaged with additional working lumens of the endoscope 14 and/or may be swapped according to the stage of the procedure for the components 14, 16. The additional components may include catheters for inverting diverticula into the intestinal lumen, transmural suturing/clipping devices, detachable ligating devices, and polypectomy snares.

The outer tube 12 may have a length of about eighty to one hundred centimeters from its open proximal end 12a to its open distal end 12b, and may have a constant outside diameter “OD” of about twenty millimeters. With this length, the tube 12 can extend completely from the anal orifice of an adult patient to the caecum, as opposed to ending at the sigmoid colon, thereby providing a pathway for advancing an instrument such as an endoscope all the way from the natural orifice to the caecum. As set forth further below, the outer tube 12 may be made from a transparent polyvinylchloride (PVC) plastisol material with stainless steel reinforcing coil embedded therein. The coil may have a diameter of about sixteen mils. The transparent plastic body permits visualization of tissue that may have been retracted into the tube 12 as well as illumination through the tube 12 to illuminate the surgical area.

The components 14, 16 may extend through respective working lumens of the outer tube 12, but in the embodiment shown the components 14, 16 extend through respective working lumens of a flexible insert 22 that, except for its lumens, substantially fills the outer tube 12, both radially and longitudinally. That is, the insert 22 is closely received in the large single central opening of the outer tube 12, and may be replaced by a substitute insert 24 that has three working lumens 26 as shown. Any number of inserts may be provided, so that a user can easily configure the assembly 10 to have as many or as few working lumens as desired, with desired sizes for the working lumens, simply by selecting the appropriate insert and engaging it with the outer tube 12.

Because only two instruments are intended to be used in the non-limiting application shown, the insert 22 has only two working lumens as shown which advantageously closely receive their respective components 14, 16. The insert 22 may otherwise be solid except for the working lumens.

As also shown in FIG. 1, the open distal end of the outer tube 12 may be filled by a distal plug 28 that can be tapered and that can also be hollow if desired so that, e.g., the endoscope 14 can extend out of it. The distal plug 28 may be separate from the insert 22 or may be made integral therewith, and may be made of a soft thermoplastic such as urethane that does not absorb water. The distal plug 28 preferably fits loosely in the catheter and glides easily over a scope. A lubricant 30 such as K-Y jelly or other hydrogel may coat the outside of the outer tube 12 and the scope 14 to facilitate placement in the colon. The lubricant 30 may also coat one or more of the lumens described herein.

Another non-limiting method is to line the inside of the outer tube 12 with a first layer of lubricious material such as Teflon or other compounds such as PVC plastisol to reduce friction and ease scope movement.

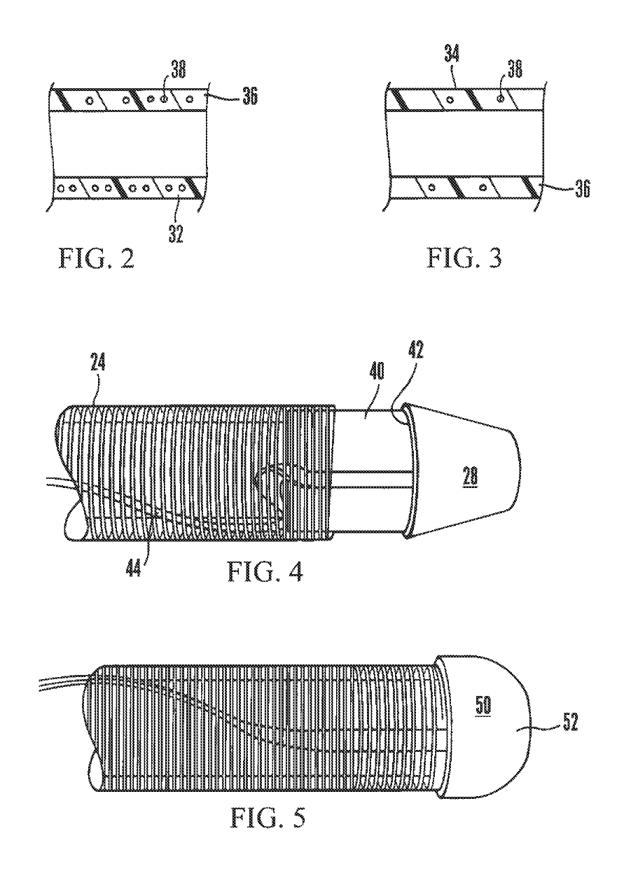

FIGS. 2 and 3 show that the outer tube 12 may have differing flexibility along different segments. More specifically, the proximal portion 32 of the outer tube 12 may be stiffer, i.e., less flexible, than the distal-most segment 34, which may be around ten to fifteen centimeters in length. The distal segment 34 is relatively more flexible for initiating turns, while the proximal portion 32, while retaining some flexibility, retains adequate column strength and hoop strength for torque control and to prevent kinking and collapse.

In one implementation, the outer tube 12 includes a cylindrical sheath 36 made of, e.g., polyvinylchloride (PVC) plastisol, in which is embedded a stainless steel reinforcing coil 38. As shown in cross-reference to FIGS. 2 and 3, in the proximal portion 32, the coil 38 has less spacing between adjacent loops than it has in the distal segment 34. That is, the distal segment 34 has fewer turns of coil per inch than the proximal segment. Furthermore, the sheath 36 of the proximal portion 32 may be harder than it is around the distal segment 34. In one non-limiting implementation the sheath 36 has a 60 Shore A hardness around the proximal portion 32 and a 45 Shore A hardness around the distal segment 34.

One non-limiting method for making the tube 12 is to wind the coil onto a mandrel in the desired number(s) of turns per inch, then dip the mandrel into liquid plastic for each of the proximal and distal segments, then bond the segments together. Or, when the same hardness plastic but differing thicknesses are used, the mandrel with coil is dipped into the liquid plastic in a way that results in differing thicknesses of plastic, rendering the proximal and distal segments unitary with each other.

FIG. 4 shows additional details of the distal segment 34 of the outer tube 12. As shown, the distal plug 28 is frusto-conical in shape, and may include a cylindrical stalk portion 40 that can be advanced into the open distal end of the outer tube 12 to engage the interior of the distal segment 24 in an interference fit. FIG. 4 depicts the plug 28 pushed part way out of the tube 12, it being understood that a lip 42 formed between the frusto-conical and cylindrical portions can abut the periphery of the distal end of the tube 12 when the plug 28 is positioned as intended.

A user can advanced an instrument through the tube 12 to dislodge the plug 28 from the tube 12 if desired, to, e.g., establish access to the bowel through the now-open distal end of the tube. To this end, a tether 44, which can be seen through the transparent wall of the tube 12, can be attached to the plug 28, so that a gripper or forceps or other instrument can be used while the tube 12 is removed from the patient to retrieve the plug 28 from the patient. Alternatively, the tether can be attached to the tube so that plug retrieval is accomplished when the tube is withdrawn from the colon.

FIG. 5 shows an alternate distal plug 50 that can have a rounded shape as shown, tapering down to a substantially flat distal face 52. Both distal plugs in FIGS. 4 and 5 prevent tissue entrapment and provide a smooth leading edge for ease of pushing the outer tube 12 through the bowel.

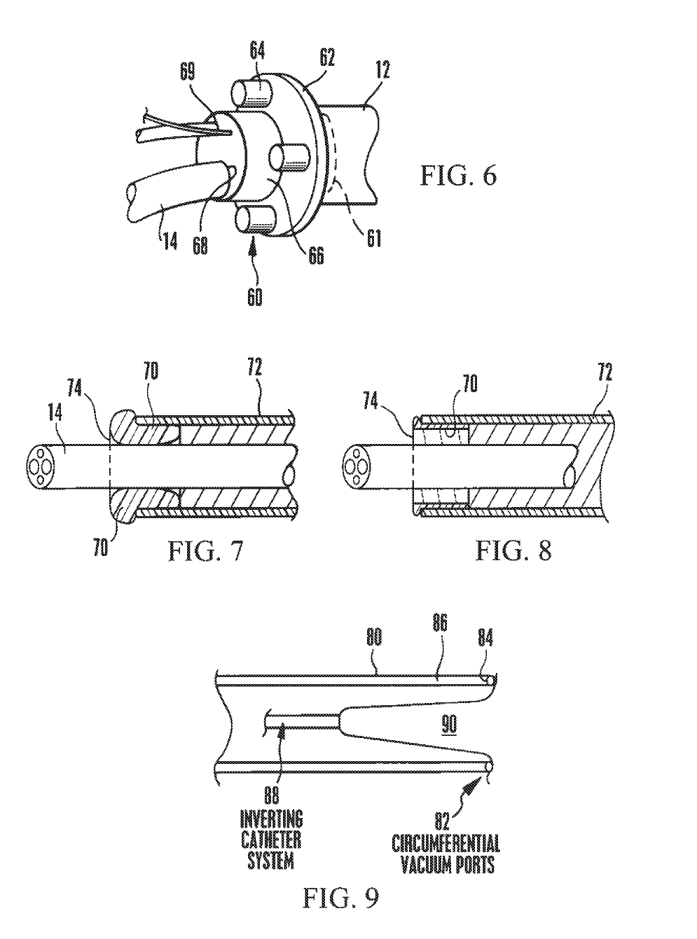

FIG. 6 shows a proximal handle 60 that can be advanced into the open proximal end (single main lumen) of the outer tube 12 to adapt the open proximal end for a smaller instrument. It is to be understood that the handle 60 may simply include a short cylindrical plug 61 (shown in phantom in FIG. 6) that is engaged with the tube 12 in an interference fit and/or glued thereto, or it may be part of or attached to one of the above-discussed elongated inserts that extend substantially the entire length of the tube 12. One of the above-discussed inserts may be advanced through the proximal handle 60 into the outer tube 12.

In the embodiment shown, the handle 60 includes a disk-shaped flange 62 that is radially larger than the tube 12. Four gripping pins 64 extend proximally away from the disk 62 and are substantially equidistantly spaced around the periphery of the disk. A person can grasp the pins 64 to rotate the outer tube 12 as desired.

The handle 60 is hollow, and a disk-shaped adapter cap 66, which may be flexible plastic, is engaged with the disk 62. To this end, the adapter cap 66 may be formed on its distal face with circular protrusions that fit tightly within corresponding grooves in the disk 62.

As shown, the proximal cap 66 forms at least one lumen 68 that is smaller than the large main lumen of the tube 12. The lumen 68 of the proximal cap 66 is sized to fit snugly around, e.g., the endoscope 14 as shown, which may also function as a gas insufflation catheter that may be connected to a source of bowel insufflating fluid or the vacuum catheter 16 shown in FIG. 1. In either case, a seal is formed around the catheter and small lumen 68, so that, e.g., if insufflating gas is infused into the bowel through a catheter extending through the cap 66, it will not easily leak out of the outer tube 12. Similarly, if the vacuum catheter 16 is advanced through the cap 66 to the distal end of the outer tube 12 to evacuate tissue into the tube 12, the vacuum is maintained by the close cooperation of the small lumen 68 with the exterior wall of the vacuum catheter 16. Additional small holes 69 may be provided in the cap for closely receiving guidewires and other components.

FIGS. 7 and 8 show an alternate distal plug 70 that is engaged with the interior of an outer tube 72 which in all other respects may be identical to the outer tube 12 discussed above. The distal plug 70 may be a toroidal-shaped inflatable structure such as a balloon, and can be adhered to the inside surface of the tube 70 to circumscribe the open distal end 74 of the tube. An instrument such as the above-mentioned endoscope 14 can be advanced through the distal plug 70 which, when inflated, provides a seal between the instrument and the outer tube 72. The balloon can have a variable tightness around the endoscope depending on user-established inflation pressure. A small inflation tube or lumen (not shown) can be provided along the length of the outer tube 72 to provide a pathway for infusing and removing inflation fluid to the plug 70. The plug 70 may be inflated as desired as shown in FIG. 7 to accommodate the diameter of the particular instrument being advanced through the outer tube 72 and then deflated as shown in FIG. 8 to facilitate moving the instrument through the plug 70, into or out of the patient.

FIG. 9 shows an outer tube 80 that in all essential respects is identical to the tubes discussed above, except that its circular distal face 82 establishes a circumferential vacuum port. The face 82 may be concave or V-shaped as shown, and one or more vacuum holes 84 can establish fluid communication between the face 82 and a vacuum lumen 86 that can extend the length of the tube 80 and that can communicate with, e.g., the source of vacuum 17 shown in FIG. 1. With this structure, the tube 80 can be positioned against tissue and a vacuum established around its distal end, to facilitates for example, an inverting catheter system 88 to invert tissue 90 into the tube 80. Details of the inverting catheter system 88 are set forth in one or more of the above-referenced patent applications.

The vacuum seal provided by the circumferential vacuum distal end of the tube 80 stabilizes the tube 80 at the target site and provides a closed chamber for cleansing the surgical site, which is now isolated from the rest of the bowel. It also limits exposure of colonic tissue in the event of unintended perforation.

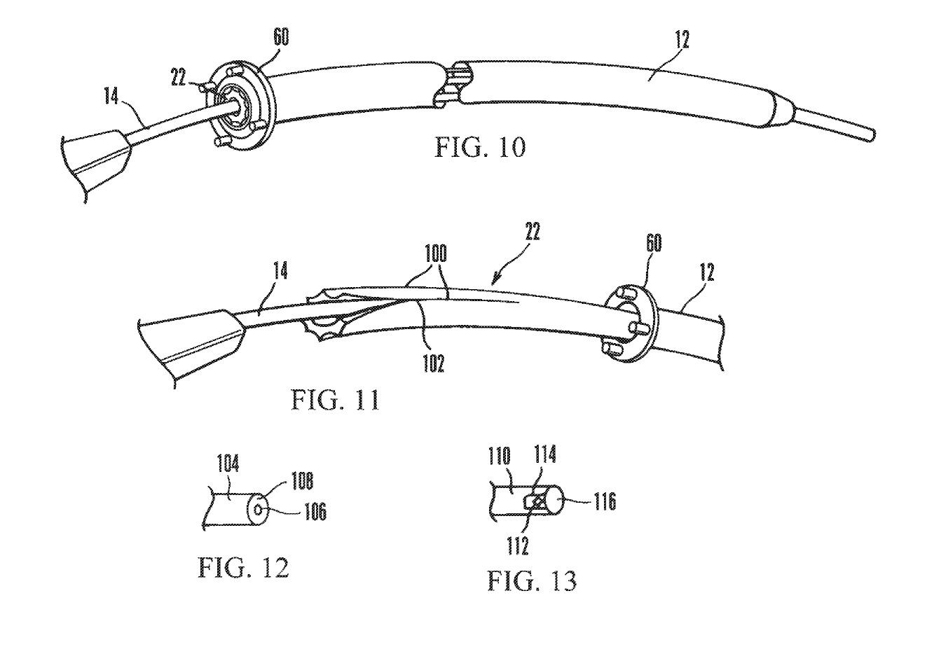

FIGS. 10 and 11 show that in non-limiting implementations, the insert 22 shown and discussed above may be formed with plural longitudinal ribs 100 that are radially spaced around the otherwise cylindrical body of the insert and that rise radially therefrom, to facilitate engagement of the insert 22 with the outer tube 12. The insert 22 may also be formed with a longitudinal slit 102 as shown, through which access to the interior of the insert 22 may be gained. The slit 102 preferably is biased to be closed.

FIG. 12 shows that a forward-viewing endoscope 104 include optics 106 that are disposed for imaging space directly in front of the distal end 108 of the endoscope 104. In contrast, FIG. 13 shows that a side-viewing endoscope 110 include optics 112 that are disposed for imaging space through, e.g., a window 114 in the side of the endoscope 110, i.e., space that is lateral to the distal end 116 of the endoscope 110. Or, the side-viewing endoscope may have a distal end bent ninety degrees with respect to the axis of the scope, with optics disposed to image space out of the bent distal end, i.e., space that is lateral to the axis of the scope relative to the organ in which the scope is positioned.

With the above structure, the following non-limiting procedure may be performed. The forward-viewing endoscope 104 may be advanced through the anal orifice of a patient to the caecum. Then, the tube 12 may be advanced over the endoscope 104, and the endoscope 104 removed from the patient. The side-viewing endoscope 110 may then be easily advanced through the tube 12 to the caecum. It will readily be appreciated that the tube 12 facilitates advancing the side-viewing endoscope 110 into the bowel, which would otherwise be rendered more difficult without the tube 12 since the side-viewing scope 110 cannot easily provide a view ahead of where the scope is being pushed. Similarly, the introduction of bulky endoscopic ultrasound probes or stapling devices is made possible.

The side-viewing endoscope 110 is useful for, e.g., viewing for conducting a natural orifice appendectomy in accordance with one or more of the above-referenced patent applications. It may now be appreciated that the removable distal plug 28 may be pushed off the end of the tube 12 when it is desired to invert excised tissue such as an appendix into the tube 12 using a vacuum, with the tube 12 thus providing an advantageously large megalumen in which to draw the tissue.

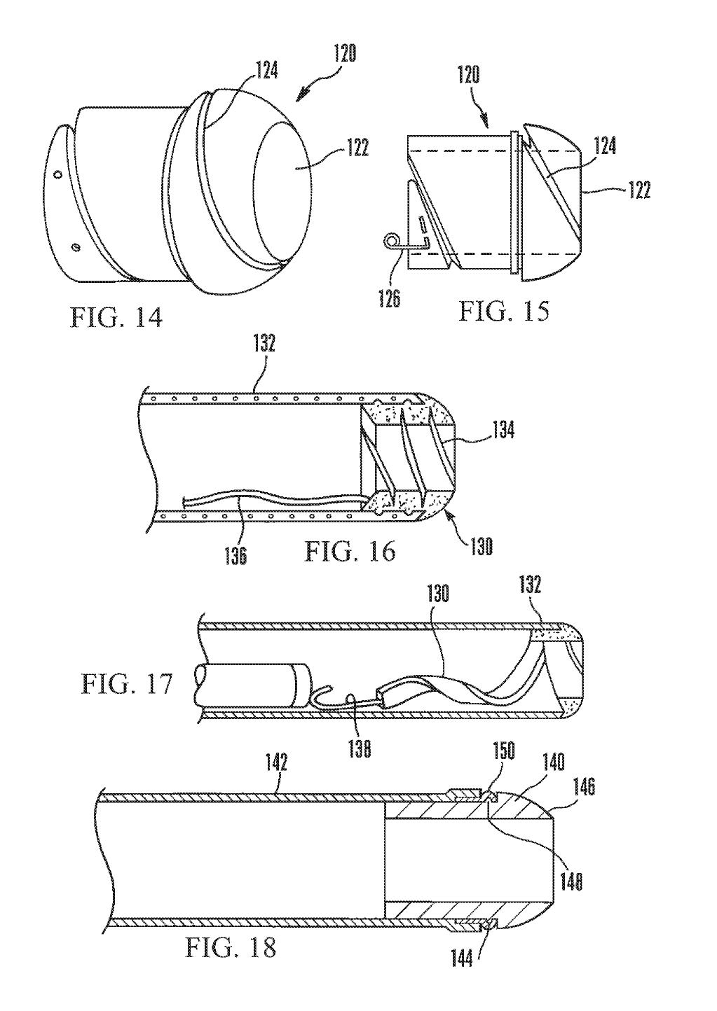

FIGS. 14 and 15 show an alternate distal plug 120 that can have a rounded shape as shown, tapering down to a substantially flat distal face 122. The plug 120 fits snugly into the distal end of an outer tube such as any of those described above.

As shown, the body of the plug 120 has a spiral score 124 formed on its outer surface. The outer score, which defines a wall thickness that is less then the thickness of the wall defined by the remainder of the plug body, facilitates material separation along its length.

As best shown in FIG. 15, a rigid metal hook 126 may be embedded in the plug body in a proximal area of the body by, e.g., overmolding the plug body onto the hook 126. The hook can be grasped by a tool, e.g., a polypectomy snare which is introduced through the outer tube to engage the hook, and the snare pulled proximally to tear the plug body in accordance with disclosure below to facilitate removing it proximally through the outer tube.

With more specificity, an alternate plug 130, which may be made of molded silicone or thermoplastic elastomer, is shown in FIGS. 16 and 17 snugly engaged with the open distal end of an outer tube 132. The plug 130 shown in FIGS. 16 and 17 is in all essential respects identical to the plug 120 shown in FIGS. 14 and 15 except the plug 130 in FIGS. 16 and 17 has a spiral score 134 formed on its inner surface, and instead of a rigid metal hook, the distal end of a cloth or other flaccid tether 136 is embedded in or adhered to the plug 130 to establish, in lieu of the wire hook described above, a disruption tool to cause material separation along the spiral score. The tether 136 is grasped and pulled by, e.g., a snare 138 (FIG. 17) which results in the deformation by material separation of the plug 130 shown in FIG. 17, in which the plug may be easily retrieved through the tube 132.

FIG. 18 shows a plug 140 that may be established by any of the plugs herein (with material separation structure omitted for clarity) snugly fitting in a distal end of an outer tube 142. As shown, the plug 140 is formed with a flange-like retention tongue 144 proximal to a distal tip portion 146 of the plug, and the tongue 144 is closely received in a complementarily-shaped groove 148 of a metal or hard plastic annular tip ring 150 that is positioned at the distal end of the outer tube 142. The tip ring 150 may be molded or glued to the tube 142. In any case, the tip ring 150 cooperates to more reliably hold the distal plug 140 in place in the open distal end until such time as it is desired to remove the plug.

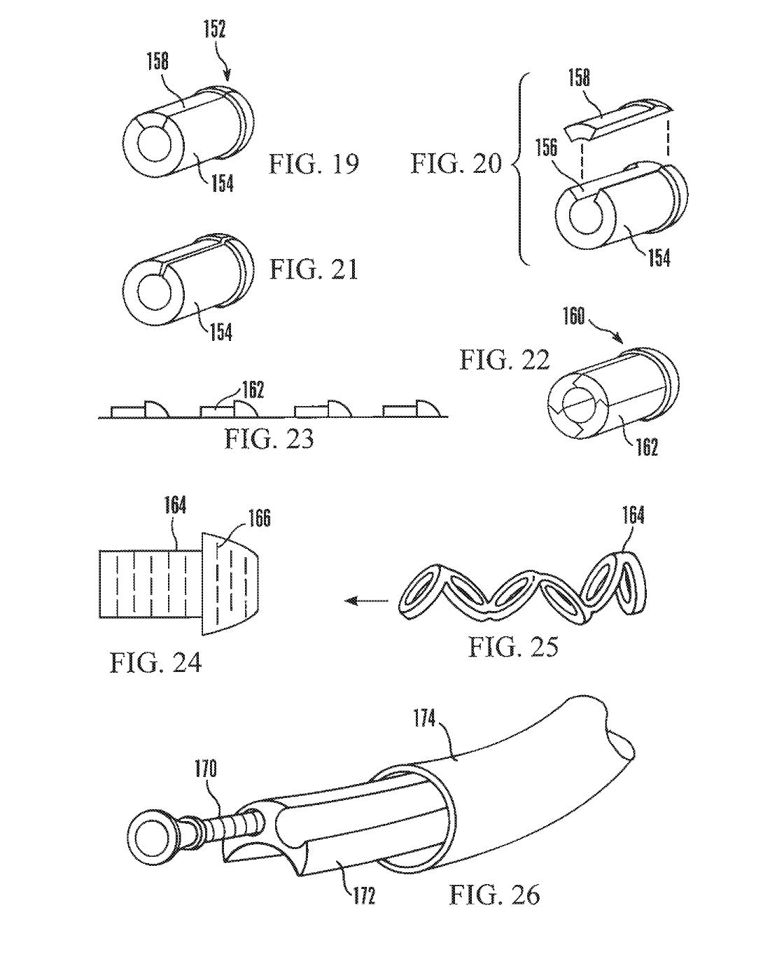

FIGS. 19-21 show an alternate distal plug 152 that is formed with a main body portion 154 defining a cylinder with axial portion of the cylinder cutout to form an opening 156. A separate keystone portion 158 fits snugly in the cutout opening 156 as shown. The keystone portion 158 is removable from the cutout opening 156 (using, e.g., a snare or other tool) to facilitate radially collapsing the main body portion 154 as shown in FIG. 21 to permit removal of the main body portion through the outer tube. It will readily be appreciated that with the keystone portion 158 in place as shown in FIG. 19, the plug 152 defines a relatively large radius to snugly fit within the tube, whereas with the keystone portion 158 removed as shown in FIG. 21, the radius of the main body portion is relatively smaller, permitting removal of the plug through the tube.

FIGS. 22 and 23 show another alternate plug 160 which includes plural (e.g., four) substantially identically-sized axial segments 162 that can be joined side by side as shown best in FIG. 22 to define a cylindrical configuration. The segments 162 can be connected together by a connector 164 such as a tether string. The segments 162 can be separated from each other as shown in FIG. 23 using, e.g., a snare or other tool to facilitate removal of the segments through the outer tube.

FIGS. 24 and 25 show another alternate plug 164 formed with plural perforations 166 to facilitate deforming the plug to the configuration shown in FIG. 25. For instance, a tool such as a grasper or snare can be used to grip the proximal end of the plug and pulling on the tool can disrupt the material of the plug body to break it into the configuration shown in FIG. 25.

FIG. 26 shows that an elongated axially rigid stiffener 170 can be engaged with a multi-lumen insert 172 that may be substantially identical to any of the inserts described above. The stiffener 170 fits tightly in one of the lumens of the insert 172 to push the insert 172 into the main lumen of an elongated flexible outer tube 174 such as any of the outer tubes described above.

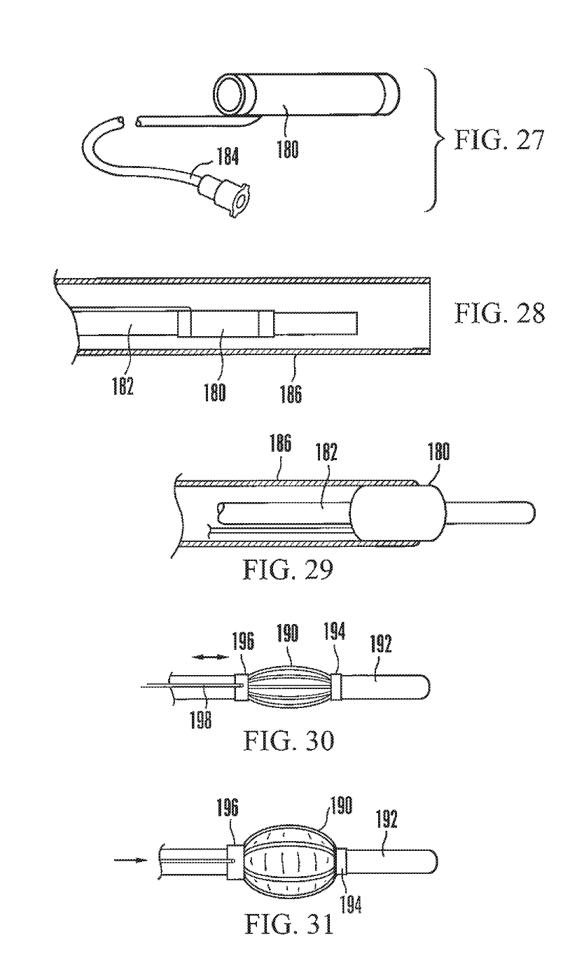

FIGS. 27-31 show alternate structures for selectively closing the open distal end of any of the outer tubes described above with the structures being deformable to permit removing them proximally through the outer tube. Beginning with FIGS. 27-29, a balloon 180 may be configured in a deflated configuration and may be engaged with an outer surface of an endoscope 182 as shown in FIG. 28. The endoscope 182 may bear an inflation lumen or tube 184 (FIG. 27) terminating in an inflation port communicating with the interior of the balloon 180 to provide a pathway for fluid communication to inflate and deflated the balloon. With this structure, the balloon 180 may be placed in the deflated configuration to advance it to and retrieve it from the open distal end of an outer tube 186 (FIG. 28) and may be placed in the inflated configuration (FIG. 29) to hold it snugly within the tube 186 to block the distal end of the tube.

Instead of a balloon, FIGS. 30 and 31 show that a metal or plastic expandable mesh 190 may be positioned on a delivery tube 192 such as an endoscope that is deliverable through an outer tube. The distal part of the mesh 190 is affixed to a fixed ring 194, whereas the proximal part of the mesh is affixed to a slide ring 196 that can be moved by means of a control wire or rod 198 to move the mesh from a radially smaller configuration (FIG. 30), wherein the mesh 190 can be advanced easily through an outer tube, to a radially enlarged configuration (FIG. 31), wherein the mesh substantially snugly fills the open distal end of an outer tube. The mesh may be biased to the enlarged configuration and moved to the small configuration by pulling the wire 198.

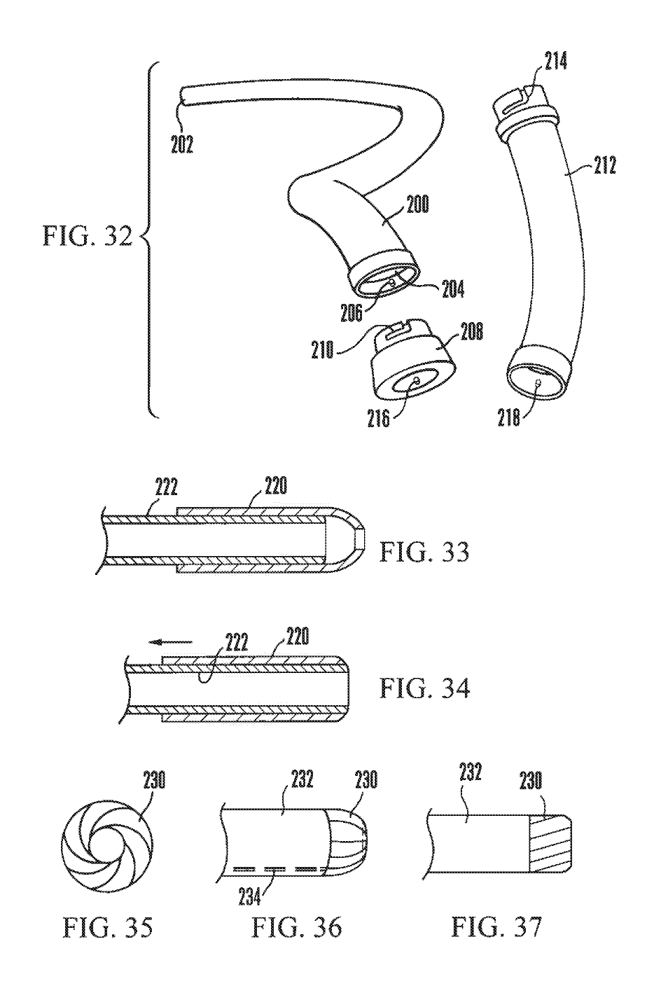

FIG. 32 shows an outer tube 200 that has an open distal end 202 and an open proximal end 204 with locking structure 206 such as but not limited to bayonet lock detents on its inner surface. An annular metal or plastic connector stub 208 with distal locking structure 210 such as an L-shaped bayonet lock channel can be removably engaged with the structure 206 of the tube 200. In turn, an elongated extension tube 212 with distal locking structure 214 such as an L-shaped bayonet lock channel can be removably engaged with proximal locking structure 216 of the stub 208 coaxially therewith to add working length to the assembly when, for example, the outer tube 200 has been advanced into a patient through a natural orifice and found to be insufficiently long. If desired, the extension tube 212 can include proximal locking structure 218 so that it, too, may be extended in accordance with principles above.

FIGS. 33 and 34 show a distal flexible nose tube 220 can surround an outer tube 222 and is movable to a blocking configuration (FIG. 33), wherein the distal portion of the nose tube 220 extends beyond the open distal end of the outer tube and is materially biased to extend radially inwardly from the wall of the outer tube 222. When the nose tube is retracted as shown in FIG. 34, owing to its flexibility substantially the entire nose tube is positioned flush against an outer wall of the outer tube.

FIGS. 35-37 show an iris-type metal or plastic distal tip 230 surrounding an outer tube 232 and being movable to a blocking configuration (FIG. 36), wherein the tip extends beyond the open distal end of the outer tube and at least a portion of the tip extends radially inwardly from a wall of the outer tube. The tip 230 also has a retracted configuration (FIG. 37) that can be established by pulling on the tip 230 using a trip wire 234. In the retracted configuration, substantially the entire tip is positioned flush against the outer wall of the outer tube.

It will be appreciated that the tip 230 may be made of rigid bard segments that are materially biased to fold toward each other as shown in FIG. 36, and that can be urged away from each other by pulling the tip distally on the tube as shown in FIG. 37.

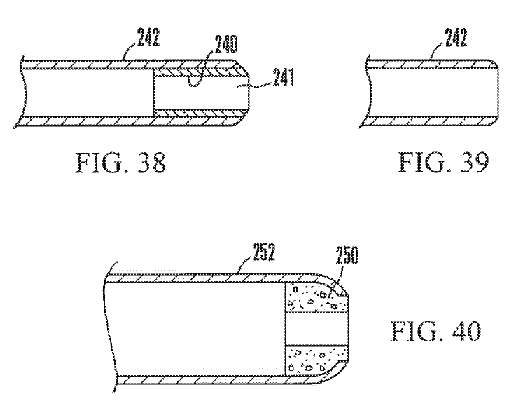

FIGS. 38 and 39 show a plug 240 that is annular and cylindrical for receiving, e.g., an endoscope therein and that may have an open distal end 241 as shown or a closed distal end. The plug 240 is tightly received in the inner diameter of the distal end of an outer tube 242 to close or partially close the distal end of the outer tube, and as shown in FIG. 242 the plug 240 can be grasped and retrieved proximally through the outer tube 242.

FIG. 40 shows a plug 250 that may be made of conformable material such as urethane foam. The plug 250 may be annular for receiving, e.g., an endoscope therein and may taper radially inwardly toward its distal end as shown. The plug 250 is tightly received in the inner diameter of the distal end of an outer tube 252 and owing to its conformability, can accommodate tools (such as endoscopes) of varying radial sizes.

While the particular CLOSURES FOR OUTER TUBE FOR NATURAL ORIFICE SURGERY is herein shown and described in detail, it is to be understood that the subject matter which is encompassed by the present invention is limited only by the claims.

What is claimed is:

1. Apparatus for engaging an end of a tube, comprising:
a plug having a body and a spiral score on an outer surface of the body to facilitate material separation therealong; and
a disruption tool configured to engage the body to cause material separation along the spiral score.

2. The apparatus of claim 1, wherein the disruption tool includes a hook embedded in the body and graspable by a snare to impart tensile force to cause the material separation.

3. The apparatus of claim 1, wherein the tube is an outer tube configured to slidably bear instruments for natural orifice surgery.

4. The apparatus of claim 3, comprising the tube.

5. The apparatus of claim 4, wherein the plug fits snugly in the end of the tube.

6. Assembly comprising:
an outer tube configured to bear one or more instruments for natural orifice surgery, the outer tube defining an open distal end;
a plug closely received in the open distal end and proximally removable therefrom; and
means for deforming the plug to permit the plug to be retrieved through the outer tube.

7. The assembly of claim 6, wherein the means for deforming includes a rigid hook pullable to cause the plug to tear.

8. The assembly of claim 6, wherein the plug includes a body having a first portion materially weaker than a second portion of the body.

9. The assembly of claim 8, wherein the first portion includes a wall defining a first thickness, the second portion including a wall defining a second thickness greater than the first thickness.

10. The assembly of claim 9, wherein the first portion includes at least one score.

11. The assembly of claim 10, wherein the first portion is characterized by a spiral score formed in the body.

12. The assembly of claim 11, wherein the score is formed on an inside surface of the body.

13. The assembly of claim 11, wherein the score is formed on an outside surface of the body.

14. The assembly of claim 6, wherein the plug is formed with a flange-like retention tongue proximal to a distal tip portion of the plug, and the tongue is received in a groove of a tip ring positioned at the distal end of the outer tube.

15. The assembly of claim 6, wherein the plug is formed with a main body portion defining a cylinder with axial cutout and a keystone portion fitting snugly in the cutout, the keystone portion being removable from the cutout to facilitate radially collapsing the main body portion to permit removal thereof through the outer tube.

16. The assembly of claim 6, wherein the plug includes plural axial segments joinable to define a cylindrical configuration, the segments being connected together by a connector and being separable from each other to facilitate removal of the segments through the outer tube.

17. The assembly of claim 6, wherein the plug is formed with plural perforations to facilitate deforming the plug.

18. The assembly of claim 6, wherein the plug is an inflatable balloon positioned on an endoscope deliverable through the outer tube.

19. The assembly of claim 6, wherein the plug is an expandable mesh positioned on an endoscope deliverable through the outer tube.

20. The assembly of claim 6, wherein the plug is an inner body closely received in the outer tube at a distal end thereof.

21. The assembly of claim 20, wherein the body is deformable and is cylindrical.

22. The assembly of claim 20, wherein the body is deformable foam.

23. The assembly of claim 22, wherein the body defines a tapered distal segment.

24. An assembly for natural orifice surgery, comprising:
an elongated flexible outer tube defining a single main lumen, wherein the outer tube defines an open distal end; and
a distal flexible nose tube surrounding the outer tube and being movable from a blocking configuration, wherein the nose tube extends beyond the open distal end of the outer tube and at least a portion of the nose tube extends radially inwardly from a wall of the outer tube, and a retracted configuration, wherein substantially the entire nose tube is positioned flush against an outer wall of the outer tube.

25. An assembly for natural orifice surgery, comprising:
an elongated flexible outer tube defining a single main lumen, wherein the outer tube defines an open distal end; and
an iris-type distal tip surrounding the outer tube and being movable from a blocking configuration, wherein the tip extends beyond the open distal end of the outer tube and at least a portion of the tip extends radially inwardly from a wall of the outer tube, and a retracted configuration, wherein substantially the entire tip is positioned flush against an outer wall of the outer tube.

26. An assembly for natural orifice surgery, comprising:
an elongated flexible outer tube defining a single main lumen, wherein the outer tube defines an open distal end;
a connector stub removably engageable with a proximal end of the outer tube coaxially therewith; and
an elongated extension tube removably connected to the stub coaxially therewith to add working length to the assembly.

27. The assembly of claim 26, wherein the tubes are removably engageable with the stub using bayonet locks.

28. An assembly for natural orifice surgery, comprising:
an elongated flexible outer tube defining a single main lumen;
an insert slidably engageable with the main lumen and defining at least one sub-lumen, the insert substantially radially filling the main lumen, the sub-lumen configured for slidably receiving an endoscope; and
an axially rigid stiffener engageable with the insert to push the insert into the main lumen.
